(19) United States
(12) Patent Application Publication
(10) Pub. No.: US 2020/0000351 A1
(43) Pub. Date: Jan. 2, 2020

(54) SMART WATCH ACCESSORY FOR IMPROVED PPG HEART RATE READING

(71) Applicant: Finnovate Group LLC, Solana Beach, CA (US)

(72) Inventor: Kari Kristian Rauhala, Solana Beach, CA (US)

(21) Appl. No.: 16/001,846

(22) Filed: Jun. 6, 2018

Related U.S. Application Data
(60) Provisional application No. 62/516,016, filed on Jun. 6, 2017.

(51) Int. Cl. A61B 5/024; A61B 5/00; A61B 5/01

(52) U.S. Cl. CPC A61B 5/02427; A61B 5/681; A61B 5/01; A61B 5/002; A61B 5/6831

(57) ABSTRACT

An apparatus include an elastomeric barrier that circumscribes a PPG sensor on an underside of a housing of a wearable device. The elastomeric barrier is configured to maintain a position of the housing on the human's skin to fix the PPG sensor at a location on the human's skin to sense the function of a human organ at the location on the human's skin. The apparatus can include an enclosure that attaches to and at least partially encloses the housing of the wearable device. The apparatus can further include a band with tightly spaced ridges, and a ratchet configured to apply micro-sized adjustments for applying tension by the band to secure the wearable device to the location on the human's skin.

### CROSS-REFERENCE TO RELATED APPLICATIONS

This application claims priority to and the benefit of U.S. Provisional Application No. 62/516,016, filed Jun. 6, 2017, which is incorporated herein by reference in its entirety.

### BACKGROUND

Current smart watches, such as those produced by Apple®, Garmin®, and Fitbit®, and fitness bands that have a built in heart rate monitor, all use a form of heart rate monitoring known as a photoplethysmogram (PPG). A PPG is an optically obtained plethysmogram, which is a volumetric measurement of an organ. A PPG is often obtained by using a light emitting diode (LED) and a pulse oximeter that illuminates the skin and measures changes in light absorption. A conventional pulse oximeter monitors the perfusion of blood to the dermis and subcutaneous tissue of the skin.

PPG sensors are very sensitive to ambient light and to anything that might vary the light reflections, such as water from an aquatic environment. For instance, when a wearer is in water, and if water is between the LED and the skin, the light can be distorted and/or reflected, and therefore the PPG based heart rate can be inaccurate. This also occurs with bright lights as the water barrier blocks ambient light.

Further PPG sensors are very sensitive to movement where the PPG sensor moves relative to the user's skin. Such movement, e.g. swimming, running, etc., introduce motion artifacts to the sensor readings. Therefore this makes the PPG readings less consistent or accurate.

Further, a wrist band used for most smart watches and fitness bands includes spaced-apart holes to adjust the fit and tightness of the watch band. It is difficult to find a perfect fit, as the process of adjusting is difficult and the wrist band may not be held in a desired place to provide a perfect fit. A wrist band that is too loose will not be able to benefit from the PPG heart rate, while a too-tight wrist band is not comfortable for the user and when the wrist band is too tight it will restrict blood flow which makes the PPG readings harder as less blood is moving through the veins under the tight sensor.

### SUMMARY

In some aspects, an apparatus to overcome the above-described limitations includes a wrist band or wrist band accessory that eliminates or significantly reduces a flow of water between PPD LEDs and a wearer's skin. In some implementations, an apparatus includes a gasket that is attached under the watch and toward the wearer's skin, leaving the PPG sensor open and an area proximate the PPG light and sensor free of water

Accordingly, the apparatus creates a barrier between the skin and the watch that prevents the water from flowing under the PPG sensor, and it also blocks any ambient light from entering between the PPG sensor and the skin, thereby improving PPG detection by the PPG sensor.

Accordingly, the apparatus creates a better and more consistent contact between the skin and the PPG sensor. An adhesive or tacky material used on the apparatus, creates a more stable connection between the skin and the wearable PPG sensors and therefore reduces and/or prevents motion artifacts during active use like swimming, running, etc.

In some aspects, the apparatus can use different lenses to help improve the PPG readings by focusing or magnifying the light into and reflecting from the tissue. The lens maybe removable and can also be shaped to increase the light focus into the user's tissue.

In another aspect, an apparatus includes a wrist band for a wearable computing device, such as a smart watch, for example, where the wearable device includes a PPG sensor. The band includes a ratcheting mechanism having small teeth that allows highly micro-sized adjustments for applying tension by the band to secure the wearable computing device to a wearer, to improve the PPG or other sensor readings that benefit from consistent, accurate, and snug placement. In some aspects the apparatus may have a pressure sensor that is used to help user select or adjust the wrist band tightness to an optimal pressure to optimize PPG readings.

In yet other aspects, an apparatus for improved volumetric measurement of a function of a human organ at a location on the human's skin, where the volumetric measurement being performed by a photoplethysmogram (PPG) sensor positioned on an underside of a housing of a wearable device, includes an elastomeric barrier that circumscribes the PPG sensor on the underside of the housing. The elastomeric barrier is configured to maintain a position of the housing on the human's skin to fix the PPG sensor at the location on the human's skin to sense the function of the human organ at the location on the human's skin

The details of one or more embodiments are set forth in the accompanying drawings and the description below. Other features and advantages will be apparent from the description and drawings, and from the claims.

### BRIEF DESCRIPTION OF THE DRAWINGS

These and other aspects will now be described in detail with reference to the following drawings.

FIGS. 1A-1C illustrate a wearable computing device, implemented as a smart watch for exemplary purposes, and having a water and light barrier to improve PPG sensing;

FIGS. 2A and 2B illustrate a wrist band with a built-in gasket, for improving PPG sensing by a wearable computing device attached to the wrist band;

FIGS. 3A and 3B illustrate illustrates a ratchet mechanism for a wrist band of a wearable device, the ratchet mechanism having micro-teeth or grooves for an improved fit to a wearer than conventional wrist bands, so as to further improve PPG sensing by wearable computing device and/or PPG sensor;

FIGS. 4A-4C illustrate an enclosure for a housing of a wearable device for improving PPG sensing.

Like reference symbols in the various drawings indicate like elements.

### DETAILED DESCRIPTION

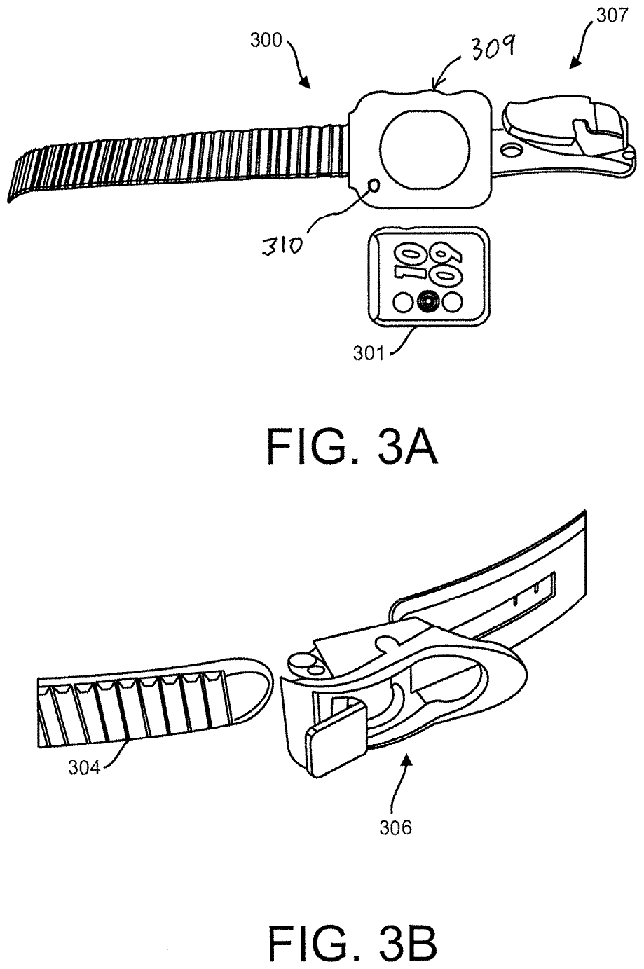

FIG. 3 illustrates a ratchet mechanism for a wrist band of a wearable device, the ratchet mechanism having micro-teeth or grooves for an improved fit to a wearer than conventional wrist bands, so as to further improve PPG sensing by wearable computing device and/or PPG sensor.

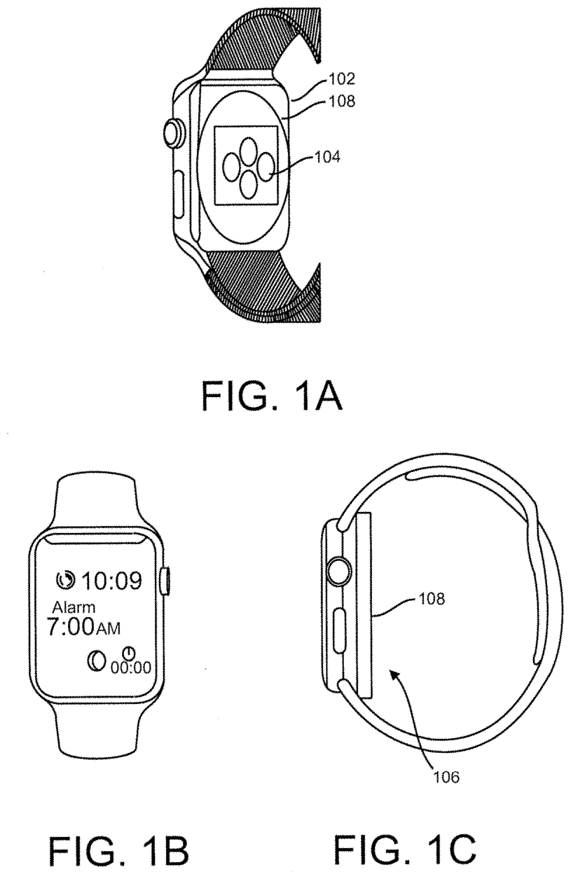

FIG. 1 shows a wearable device, implemented as a smart watch for exemplary purposes. The wearable computing device includes a housing 102 and a PPG sensor 104 on an underside 106 of the housing 102, where the underside 106 is configured to abut the wearer's skin. The PPG sensor 104 includes a set of light emitting diodes (LEDs), surrounded by a water and light barrier 108 to improve PPG sensing. For instance, the water and light barrier 108 can be formed as an elastomeric gasket, to keep water and external light out of the area between the LEDs of the PPG sensor 104 and the wearer's skin, so that the PPG sensor 104 can sense and monitor the wearer's heart rate more accurately and effectively. The gasket can be attached to the housing 102 of a watch or other wearable device by an adhesive, or can snap-in-place around the watch, or even attach to the wrist band, etc.

The water and light barrier 108 can be made materials that are soft and which easily conform to the user's arm where the wearable device is attached, while also providing a tight seal on the housing of the wearable device. For example, the gasket can be formed of any of a number of elastomers or rubbers, or any combination thereof. In some implementations, the gasket can include multiple layers, one inside another, and each layer can have a different elastomeric property and/or light-blocking property. In some implementations, the water and light barrier 108 can be formed to be hydrodynamic and reduce drag around the housing 102, particularly near the underside 106 of the housing 102.

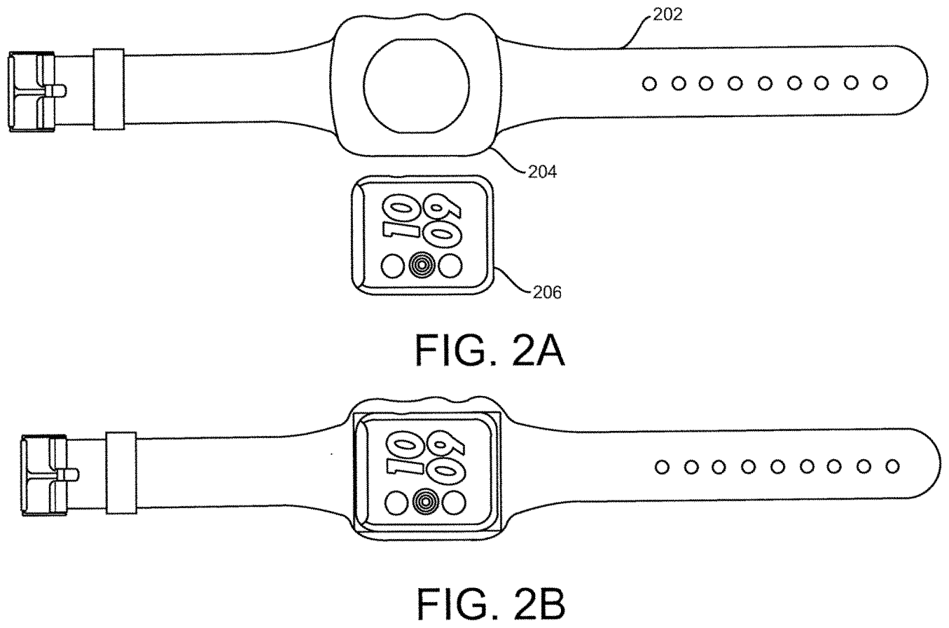

In some implementations, the water and light barrier 108 may be removable from the housing 102 of the wearable device, or can be permanently attached to the housing 102. In some implementations, the water and light barrier 108 is built into a wrist band attached to the housing 102 of the wearable device. FIGS. 2A and 2B illustrates a wrist band 202 with a built-in gasket 204 configured to surround a wearable device 206, and for improving PPG sensing by the wearable device when it is attached to the wrist band 202, as shown in FIG. 2B.

In some implementations, an apparatus can include one or more built-in pressure sensors to detect user's wrist pulse that can be transmitted to the wearable device via wireless signal, such as Bluetooth (BT) connection or other short-range wireless communication protocol. This pulse detection can be further used to enhance the PPG heart rate. The apparatus can have a built in battery that can charge the smartwatch or the PPG sensor.

In some implementations, an apparatus can include one or more built-in pressure sensors to detect pressure of the PPG sensor against the skin. The pressure readings can be sent to the wearable device via wireless signal, such as Bluetooth (BT) connection or other short-range wireless communication protocol. This pressure detection can be further used to inform the user of the optimal pressure required to optimize PPG readings. The wearable device may display indication to the user about the status of the pressure (e.g. wrist band is too loose, just right or too tight).

In some implementations, the optimal wristband tightness pressure for PPG can be measured once, and the reading can be stored digitally, or it can be manually marked on the watch band to allow the user to adjust the band to the predetermined position. The pressure sensor may also provide an audio feedback, like a snap, when the optimal pressure is achieved.

In some implementations, apparatus may have a skin temperature meter. The skin temperature meter is used to detect user's skin temperature and communicate the measurements to the smartwatch i.e. via Bluetooth. If the user's skin temperature is too low, blood circulation will be reduced and the PPG readings are more unreliable. The skin temperature and PPG reading can be displayed to the user alerting that the measurements may not be working due to low skin temperature issues.

In some implementations, the apparatus can have sensors to detect water, to detect whether water gets by the water and light barrer 108 or under or proximate the PPG LED area. The water sensor of the apparatus can detect that water is present and alert the user with a built-in LED. i.e. when water is present, a small LED light can be turned on to alert the user that heart rate readings may be compromised or inaccurate, and the user should dry the area and tighten the wrist band. In some implementations, the water and light barrer 108 can also create a waterproof seal around the entire housing, and thus making a wearable device fully waterproof, allowing a wearer to wear the wearable device in the water.

FIG. 3 illustrates a wrist band 300 for a wearable device 301, with the wrist band 300 having a ratchet mechanism 302 for a more precise and better fit around a wearer's wrist, and which may or may not be used in conjunction with a water and light barrier as described above. The ratchet mechanism 302 includes micro-teeth or grooves for an improved fit to a wearer than conventional wrist bands, so as to further improve PPG sensing by the wearable device and/or PPG sensor. In some implementations, as shown in FIG. 3, micro-teeth are formed on the wrist band 300. In other implementations, the micro-teeth can be built into the ratchet mechanism 302 and the band can be smooth. In some implementations, the wrist band 300 includes a housing or enclosure 309, which may have a pressure sensor 310 that is used to help user select or adjust the wrist band 300 tightness to an optimal pressure to optimize PPG readings.

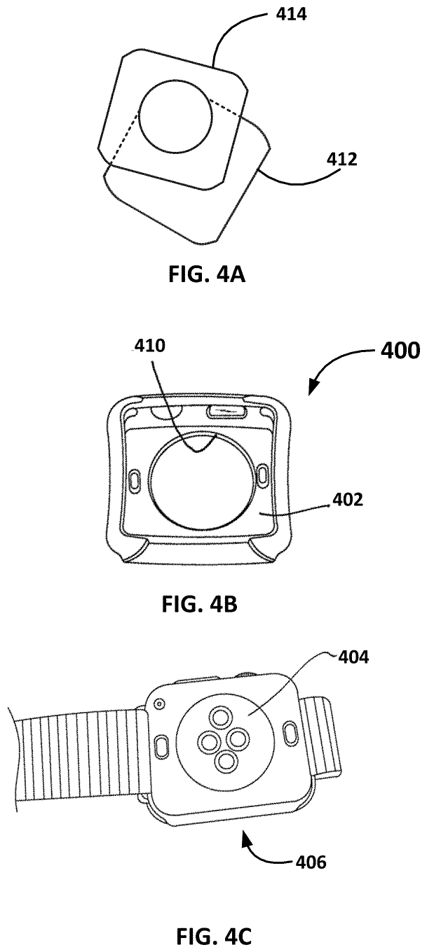

FIG. 4 illustrates an enclosure 400 that attaches to and at least partially encloses the housing 404 of the wearable device 406. The enclosure 400 can include an elastomeric barrier 410 that circumscribes the PPG sensor on the underside of the housing. The elastomeric barrier 410 can include a gasket, skirt 412, or a substantially softer, more elastic area, such as at the periphery of the elastomeric barrier 410. The elastomeric barrier 410 is configured to maintain a position of the housing on the human's skin to fix the PPG sensor at the location on the human's skin to sense the function of a human organ at the location on the human's skin, such as heartrate.

Further, the enclosure 400 can include an optical lens adapted for positioning adjacent to the PPG sensor for focusing PPG sensor-emitted light, and/or circumscribed by the elastomeric barrier for guiding PPG sensor-emitted light that is reflected back to the PPG sensor from the human's skin.

Although a few embodiments have been described in detail above, other modifications are possible. Other embodiments may be within the scope of the following claims.

1. An apparatus for improved volumetric measurement of a function of a human organ at a location on the human's skin, the volumetric measurement being performed by a photoplethysmogram (PPG) sensor positioned on an underside of a housing of a wearable device, the apparatus comprising:
an elastomeric barrier that circumscribes the PPG sensor on the underside of the housing, the elastomeric barrier being configured to maintain a position of the housing on the human's skin to fix the PPG sensor at the location on the human's skin to sense the function of the human organ at the location on the human's skin.

2. The apparatus in accordance with claim 1, further comprising an enclosure that attaches to and at least partially encloses the housing of the wearable device.

3. The apparatus in accordance with claim 2, further comprising a band configured to attach to the human's skin to position the PPG sensor at the location on the human's skin.

4. The apparatus in accordance with claim 1, wherein the elastomeric barrier is opaque to form a light barrier so as to keep external light from reaching the PPG sensor.

5. The apparatus in accordance with claim 1, wherein the elastomeric barrier forms a waterproof seal between the underside of the housing and the human's skin.

6. The apparatus in accordance with claim 1, further comprising a wrist band coupled with the housing for securing the housing to the human's skin.

7. The apparatus in accordance with claim 1, wherein the elastomeric barrier increases friction between the underside of the housing and the human's skin.

8. The apparatus in accordance with claim 1, further comprising an optical lens adapted for positioning adjacent to the PPG sensor for focusing PPG sensor-emitted light.

9. The apparatus in accordance with claim 9, wherein the optical lens is removable.

10. The apparatus in accordance with claim 1, further comprising an optical lens circumscribed by the elastomeric barrier for guiding PPG sensor-emitted light that is reflected back to the PPG sensor from the human's skin.

11. An apparatus for improved volumetric measurement of a function of a human organ at a location on the human's skin, the volumetric measurement being performed by a photoplethysmogram (PPG) sensor positioned on an underside of a housing of a wearable device, the apparatus comprising:
an elastomeric barrier that circumscribes the PPG sensor on the underside of the housing, the elastomeric barrier being configured to maintain a position of the housing on the human's skin to fix the PPG sensor at the location on the human's skin to sense the function of the human organ at the location on the human's skin, the elastomeric barrier defining an aperture; and
an optical lens fixed within the aperture defined by the elastomeric barrier.

12. An apparatus for improved volumetric measurement of a function of a human organ at a location on the human's skin, the volumetric measurement being performed by a photoplethysmogram (PPG) sensor positioned on an underside of a housing of a wearable device, the apparatus comprising:
an enclosure that attaches to and at least partially encloses the housing of the wearable device;
a band configured to attach to the human's skin to position the PPG sensor at the location on the human's skin, a proximal end of the band including a plurality of ridges tightly positioned on the band; and
a ratchet connected to distal end of the band, the ratchet configured to apply micro-sized adjustments for applying tension by the band to secure the wearable device to the location on the human's skin.
